(19) United States
(12) Patent Application Publication
(10) Pub. No.: US 2025/0228635 A1
(43) Pub. Date: Jul. 17, 2025

(54) MANIPULATION PART OF SURGICAL INSTRUMENT

(71) Applicant: LIVSMED INC., Seongnam-si (KR)

(72) Inventors: Jae Yeong LEE, Seongnam-si (KR); Hee Jin KIM, Seoul (KR); Jwa Nyun KIM, Yongin-si (KR); Chung Hyun LIM, Seoul (KR)

(21) Appl. No.: 19/016,698

(22) Filed: Jan. 10, 2025

(30) Foreign Application Priority Data
Jan. 11, 2024 (KR) 10-2024-0004934
Apr. 11, 2024 (KR) 10-2024-0048643

(51) Int. Cl. A61B 34/00; A61B 17/29; A61B 18/14

(52) U.S. Cl. CPC A61B 34/71; A61B 18/1445; A61B 2017/2912; A61B 2017/2925

(57) ABSTRACT

A manipulation part of a surgical instrument includes a handle, and an actuation manipulation part that is disposed on one side of the handle, and controls an actuation motion of an end tool including at least one jaw, wherein the actuation manipulation part includes a grip lever that rotates around an actuation rotation shaft, a grip gear that rotates around the actuation rotation shaft in response to rotation of the grip lever, a pulley gear that is engaged with the grip gear, and a grip pulley that rotates around a grip pulley rotation shaft, in response to rotation of the pulley gear, and has at least one wire connected thereto, and the grip pulley transmits power to the end tool through the at least one wire connected to the grip pulley by the rotation around the grip pulley rotation shaft.

### CROSS-REFERENCE TO RELATED APPLICATION

This application is based on and claims priority under 35 USC § 119 to Korean Patent Application Nos. 10-2024-0004934, filed on Jan. 11, 2024, and 10-2024-0048643, filed on Apr. 11, 2024, in the Korean Intellectual Property Office, the disclosure of which is incorporated by reference herein in its entirety.

### BACKGROUND

### 1. Field

The present disclosure relates to a surgical instrument, and more particularly, a surgical instrument that is operable manually to be used in laparoscopic surgery or other various surgeries.

### 2. Description of the Related Art

In medical terms, a surgery refers to curing an illness by incising, opening, or manipulating the skin, mucous membranes, or other tissues using medical instruments. In particular, an open surgery and the like, which incise and open a skin at a surgical site to treat, remodel, or remove organs in the surgical site, cause problems such as bleeding, side effects, patient's pain, scar, etc. Therefore, recently, a surgery performed by forming predetermined holes in a skin and inserting only a medical instrument, such as a laparoscope, surgical instrument, or microsurgical microscope, into the holes or a surgery using a robot, now come into the spotlight as an alternative.

A surgical instrument is a tool that a doctor uses to operate a surgical site by manipulating an end tool disposed on one end of a shaft, which passes through a hole drilled in a skin, either by a hand through a predetermined manipulation part or by using a robotic arm. The end tool disposed at the surgical instrument is manipulated to perform motions, such as rotation, gripping, cutting, etc. through a predetermined structure.

The aforementioned background technology is technical information possessed by the inventor for derivation of the present disclosure or acquired by the inventor during the derivation of the present disclosure, and is not necessarily prior art disclosed to the public before the application of the present disclosure.

### SUMMARY

The present disclosure is to provide a manipulation part of a surgical instrument that is capable of securing design flexibility and expandability with respect to a wire-pulley structure of the manipulation part, in the surgical instrument that can be actuated manually for use in laparoscopic surgery or various other surgeries.

Additional aspects will be set forth in part in the description which follows and, in part, will be apparent from the description, or may be learned by practice of the presented embodiments of the disclosure.

One embodiment of the present disclosure provides a manipulation part of a surgical instrument that includes a handle, and an actuation manipulation part that is disposed on one side of the handle, and controls an actuation motion of an end tool including at least one jaw, wherein the actuation manipulation part includes a grip lever that rotates around an actuation rotation shaft, a grip gear that rotates around the actuation rotation shaft in response to rotation of the grip lever, a pulley gear that is engaged with the grip gear, and a grip pulley that rotates around a grip pulley rotation shaft, in response to rotation of the pulley gear, and has at least one wire connected thereto, and the grip pulley transmits power to the end tool through the at least one wire connected to the grip pulley by the rotation around the grip pulley rotation shaft.

In one embodiment of the present disclosure, the actuation rotation shaft may be parallel to the grip pulley rotation shaft.

In one embodiment of the present disclosure, the grip lever may include a rotating part to which the actuation rotation shaft is coupled and which is formed in a disk shape having a certain diameter based on the actuation rotation shaft, and a grip part that is a part extending from the rotating part and gripped by a user to transmit force to the grip lever.

In one embodiment of the present disclosure, the grip gear may be formed along a circumference of the rotating part.

In one embodiment of the present disclosure, the pulley gear may be coupled to one side surface of the grip pulley to rotate integrally with the grip pulley.

In one embodiment of the present disclosure, the grip pulley may include a first grip pulley coupled to one side with respect to the pulley gear, and a second grip pulley coupled to another side with respect to the pulley gear, and the first grip pulley, the pulley gear, and the second grip pulley may be integrally coupled together, so as to rotate around a same rotation shaft.

In one embodiment of the present disclosure, when the grip lever rotates in a first direction, the grip gear may rotate in the first direction, and the pulley gear and the grip pulley may rotate in a second direction opposite to the first direction.

In one embodiment of the present disclosure, when the grip lever rotates, a rotating force transmitted to the grip pulley and a rotation angle displacement of the grip pulley may vary depending on a ratio of a diameter of the pulley gear to a diameter of the grip gear.

One embodiment of the present disclosure provides a surgical instrument that includes an end tool that includes a first jaw and a second jaw each formed to be rotatable, and is rotatable in at least two directions, a manipulation part that controls rotation of the end tool in the at least two directions, a power transmission part that includes a first jaw wire connected to the manipulation part to transmit rotation of the manipulation part to the first jaw, and a second jaw wire connected to the manipulation part to transmit the rotation of the manipulation part to the second jaw, and a connection part that has one end portion coupled to the end tool and another end portion coupled to the manipulation part, such that the manipulation part and the end tool are connected to each other, wherein the manipulation part includes a handle, and an actuation manipulation part that is disposed on one side of the handle, and controls an actuation motion of the end tool including the first jaw and the second jaw, the actuation manipulation part includes a grip lever that rotates around an actuation rotation shaft, a grip gear that rotates around the actuation rotation shaft in response to rotation of the grip lever, a pulley gear that is engaged with the grip gear, and a grip pulley that rotates around a grip pulley rotation shaft, in response to rotation of the pulley gear, and has at least one wire connected thereto, and
the grip pulley transmits power to the end tool through the at least one wire connected to the grip pulley by the rotation around the grip pulley rotation shaft.

In one embodiment of the present disclosure, the actuation rotation shaft may be parallel to the grip pulley rotation shaft.

In one embodiment of the present disclosure, the grip lever may include a rotating part to which the actuation rotation shaft is coupled and which is formed in a disk shape having a certain diameter based on the actuation rotation shaft, and a grip part that is a part extending from the rotating part and gripped by a user to transmit force to the grip lever.

In one embodiment of the present disclosure, the pulley gear is coupled to one side surface of the grip pulley to rotate integrally with the grip pulley.

In one embodiment of the present disclosure, the grip pulley may include a first grip pulley coupled to one side with respect to the pulley gear, and a second grip pulley coupled to another side with respect to the pulley gear, and the first grip pulley, the pulley gear, and the second grip pulley may be integrally coupled together, so as to rotate around a same rotation shaft.

In one embodiment of the present disclosure, the first jaw wire may include a first strand and a second strand as a pair of wires, the second jaw wire may include a third strand and a fourth strand as a pair of wires,
the first strand of the first jaw wire and the fourth strand of the second jaw wire may be connected to any one of the first grip pulley or the second grip pulley, and the second strand of the first jaw wire and the third strand of the second jaw wire may be connected to another one of the first grip pulley or the second grip pulley.

In one embodiment of the present disclosure, the grip pulley may be rotated by the grip lever in any one direction to enable an opening or closing motion of the first jaw and the second jaw.

In one embodiment of the present disclosure, at least a portion of the first jaw wire and at least a portion of the second jaw wire may be fixedly wound around the grip pulley.

In one embodiment of the present disclosure, the first jaw wire and the second jaw wire may each include a pair of wires, and one of the pair of wires may be wound around the grip pulley in a first direction while another one may be wound around the grip pulley in a second direction opposite to the first direction.

In one embodiment of the present disclosure, when the grip lever rotates in the first direction, the grip gear may rotate in the first direction, and the pulley gear and the grip pulley may rotate in the second direction opposite to the first direction, and the wire wound in the second direction may be wound around the grip pulley and the wire wound in the first direction may be unwound from the grip pulley, in response to the rotation of the grip pulley, so as to cause a rotational motion of the first jaw or the second jaw.

In one embodiment of the present disclosure, the first jaw wire may include a first strand and a second strand as a pair of wires, the second jaw wire may include a third strand and a fourth strand as a pair of wires, the first strand may be wound around the grip pulley in a first direction, while the second strand may be wound in a second direction opposite to the first direction, and the third strand may be wound around the grip pulley in the first direction, while the fourth strand may be wound in the second direction opposite to the first direction.

In one embodiment of the present disclosure, when the grip lever rotates, a rotating force transmitted to the grip pulley and a rotation angle displacement of the grip pulley may vary depending on a ratio of a diameter of the pulley gear to a diameter of the grip gear.

Other aspects, features, and advantages in addition to those described above will become apparent from the following drawings, claims, and detailed description of the disclosure.

### BRIEF DESCRIPTION OF DRAWINGS

The above and other aspects, features, and advantages of certain embodiments of the disclosure will be more apparent from the following description taken in conjunction with the accompanying drawings, in which:

FIG. 1 is a perspective view illustrating a surgical instrument to which a manipulation part is applied, according to one embodiment of the present disclosure;

FIG. 2 is a perspective view illustrating a state where a case is removed from the surgical instrument of FIG. 1;

FIGS. 3 and 4 are perspective views illustrating the manipulation part of the surgical instrument of FIG. 2;

FIG. 5 is a view briefly illustrating only a configuration of pulleys and wires constituting joints of the surgical instrument illustrated in FIG. 2;

FIG. 6 is a perspective view illustrating a yaw motion of the surgical instrument of FIG. 2;

FIGS. 7 and 8 are diagrams illustrating a configuration of pulleys and wires, which are related to an actuation motion and a yaw motion of the surgical instrument illustrated in FIG. 2, in detail for each of the first jaw and the second jaw

FIG. 9 is a perspective view illustrating a pitch motion of the surgical instrument of FIG. 2;

FIGS. 10 and 11 are diagrams illustrating a configuration of pulleys and wires, which are related to a pitch motion of the surgical instrument illustrated in FIG. 2, in detail for each of the first jaw and the second jaw

FIG. 12 is a perspective view illustrating a manipulation part of a surgical instrument according to one embodiment of the present disclosure;

FIG. 13 is a perspective view illustrating the manipulation part of the surgical instrument of FIG. 12, viewed at another angle;

FIGS. 14A and 14B are views illustrating an actuation motion of the manipulation part of the surgical instrument of FIG. 12; and

FIGS. 15A and 15B are views illustrating a manipulation part of a surgical instrument according to a modified example of the one embodiment of the present disclosure, which illustrates an actuation motion of the manipulation part.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

Description will now be given in detail of embodiments disclosed herein, with reference to the accompanying drawings. For description with reference to the drawings, the same or equivalent components may be given the same reference numerals, and a redundant description thereof will be omitted.

Since the embodiments of the present disclosure can be modified in various ways, specific embodiments will be illustrated in the drawings and specifically described in the detailed description. The effects and features of the embodiments of the present disclosure and methods for achieving the same will be clearly understood by referring to descriptions to be given below along with the drawings. However, the embodiments of the present disclosure are not limited to the embodiments disclosed below and may be implemented in various forms.

In describing the present disclosure, a detailed description of known related arts will be omitted when it is determined that the gist of the present disclosure may be unnecessarily obscured.

In the embodiments disclosed below, a singular representation may include a plural representation unless it represents a definitely different meaning from the context. Although terms such as “first,” “second,” and the like may be used to describe various components, such components should not be limited by the above terms The terms are only used to distinguish one component from another.

In the embodiments disclosed below, terms such as “include” or “has” should be understood that they are intended to indicate an existence of features or components, disclosed in this specification, and also it is not excluded in advance that one or more features or components are likewise utilized.

In the embodiments below, when a part such as a unit, area, component, etc. is said to be located on another part, it includes not only the case where the part is directly located on top of the another part, but also the case where other units, areas, components, etc. are interposed therebetween.

In the following embodiments, terms such as “connected” or “coupled” do not necessarily mean “two members being directly and/or fixedly connected or coupled,” unless otherwise specified within the context, and do not exclude the intervention of other members between the two members.

In the drawings, the sizes of components may be enlarged or exaggerated or reduced for convenience of explanation. For example, a size and a thickness of each component illustrated in the drawings are illustrative for convenience of description, and the embodiment below is not limited to the size and the thickness of the component illustrated.

Before describing a manipulation part according to one embodiment of the present disclosure, a surgical instrument will be described below.

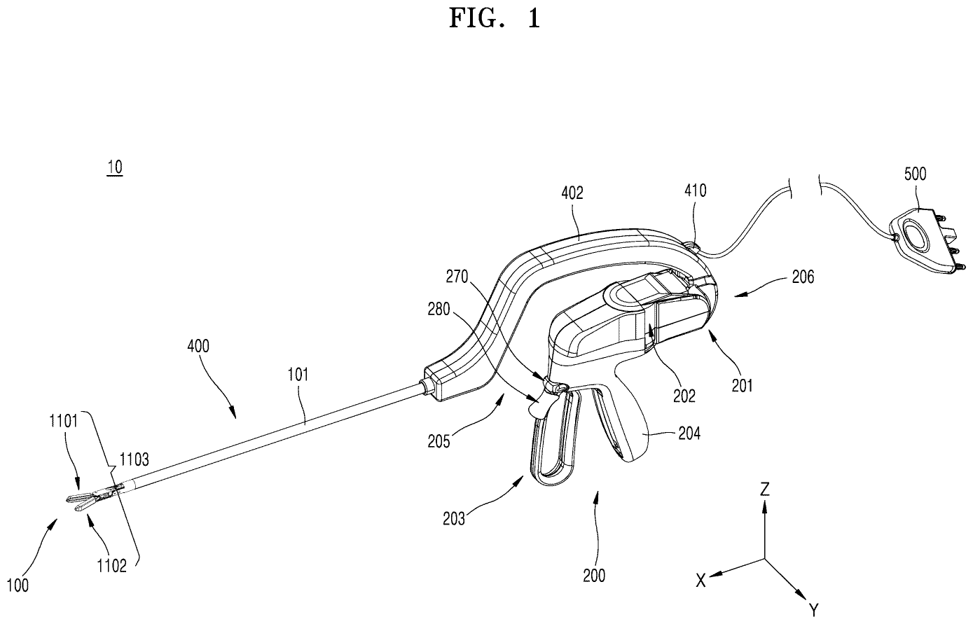

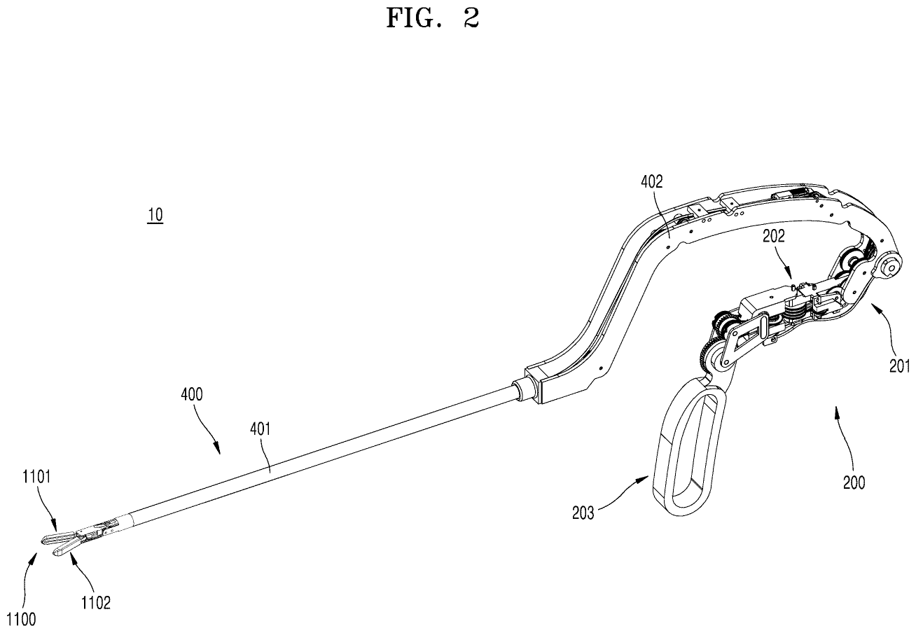

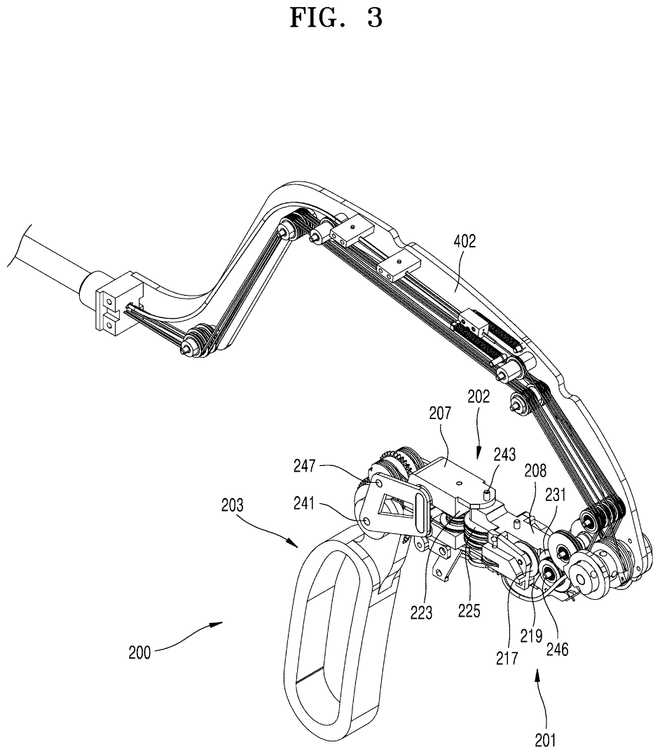

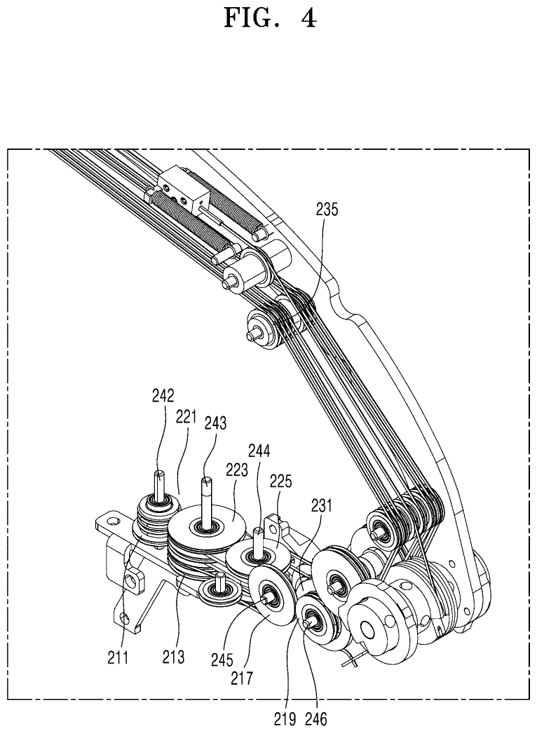

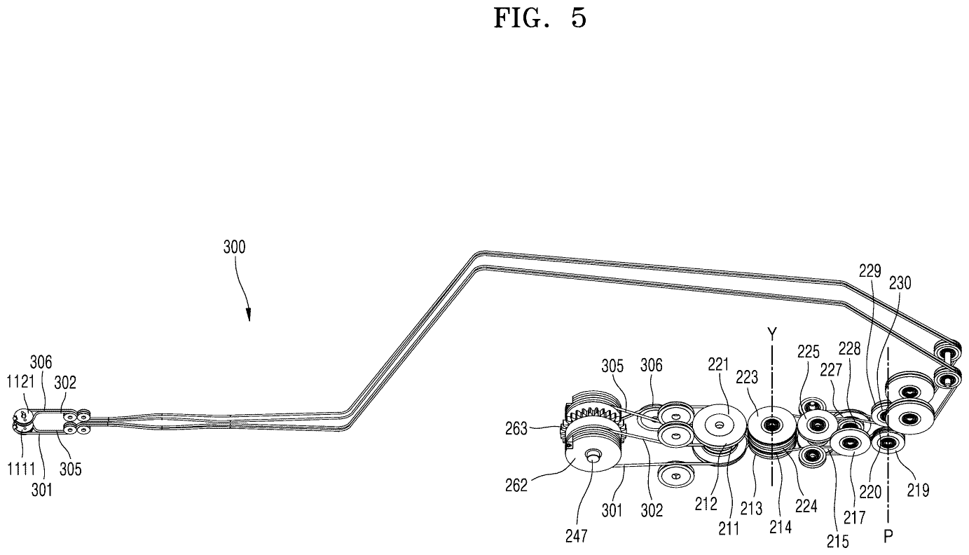

FIG. 1 is a perspective view illustrating a surgical instrument to which manipulation part is applied, according to one embodiment of the present disclosure. FIG. 2 is a perspective view illustrating a state where a case is removed from the surgical instrument of FIG. 1. FIGS. 3 and 4 are perspective views illustrating the manipulation part of the surgical instrument of FIG. 2, and FIG. 5 is a view briefly illustrating only a configuration of pulleys and wires constituting joints of the surgical instrument illustrated in FIG. 2.

Referring to FIGS. 1 to 5, a surgical instrument 10 according to one embodiment of the present disclosure includes an end tool 1100, a manipulation part 200, a power transmission part 300, and a connection part 400.

Here, the connection part 400 is formed in the shape of a hollow shaft, and one or more wires and electric wires may be accommodated therein. The manipulation part 200 may be coupled to one end portion of the connection part 400, and the end tool 1100 may be coupled to the other end portion thereof. Thus, the connection part 400 may serve to connect the manipulation part 200 and the end tool 1100. Here, the connection part 400 of the surgical instrument 10 according to one embodiment of the present disclosure includes a straight part 401 and a curved part 402. The straight part 401 is formed at a side coupled to the end tool 1100, and the curved part 402 is formed at a side to which the manipulation part 200 is coupled. As such, since the end portion of the connection part 400 at the side of the manipulation part 200 is formed to be curved, a pitch manipulation part 201, a yaw manipulation part 202, and an actuation manipulation part 203 may be formed along an extension line of the end tool 1100 or adjacent to the extension line. From another perspective, it may be said that the pitch manipulation part 201 and the yaw manipulation part 202 are at least partially accommodated in a concave portion formed by the curved part 402. Due to the above-described shape of the curved part 402, the shapes and motions of the manipulation part 200 and the end tool 100 may further intuitively match each other.

Meanwhile, a plane on which the curved part 402 is formed may be substantially the same plane as a pitch plane, that is, an XZ plane of FIG. 1. As such, as the curved part 402 is formed on substantially the same plane as the XZ plane, interference with the manipulation part can be reduced. Of course, for intuitive motions of the end tool and the manipulation part, any form other than the XZ plane may be possible.

Meanwhile, a connector 410 may be formed on the curved part 402. The connector 410 may be connected to an external power source (not illustrated). The connector 410 may also be connected to the jaws 1103 via electric wires, and may transmit, to the jaws 1103, electric energy supplied from the external power source (not illustrated). Here, the connector 410 may be a bipolar type with two electrodes, or may be a monopolar type with one electrode.

The manipulation part 200 is formed at the one end portion of the connection part 400 and provided as an interface to be directly controlled by a medical doctor, for example, a tongs shape, a stick shape, a lever shape, or the like. When the medical doctor controls the manipulation part 200, the end tool 1100, which is connected to the interface and inserted into the body of a surgical patient, performs a certain motion, thereby performing surgery. Here, the manipulation part 200 is illustrated in FIG. 1 as being formed in a handle shape that is rotatable while a finger is inserted therein, but the concept of the present disclosure is not limited thereto, and various types of manipulation parts that are connected to the end tool 1100 to manipulate the end tool 1100 may be possible.

The end tool 1100 is formed on the other end portion of the connection part 400, and performs necessary motions for surgery by being inserted into a surgical site. In an example of the end tool 1100, as illustrated in FIG. 1, a pair of jaws 1103 for performing a grip motion may be used. However, the concept of the present disclosure is not limited thereto, and various devices for performing surgery may be used as the end tool 1100. For example, a configuration of a cantilever cautery may also be used as the end tool. The end tool 1100 is connected to the manipulation part 200 by the power transmission part 300, and receives a driving force of the manipulation part 200 through the power transmission part 300 to perform a motion necessary for surgery, such as gripping, cutting, suturing, or the like.

Here, the end tool 1100 of the surgical instrument 10 according to one embodiment of the present disclosure is formed to be rotatable in at least one direction. For example, the end tool 1100 may perform a pitch motion around a Y-axis of FIG. 1 and simultaneously perform a yaw motion and an actuation motion around a Z-axis of FIG. 1.

The power transmission part 300 may connect the manipulation part 200 to the end tool 1100, transmit the driving force of the manipulation part 200 to the end tool 1100, and include a plurality of wires, pulleys, links, sections, gears, or the like.

The end tool 1100, the manipulation part 200, and the power transmission part 300 of the surgical instrument 10 of FIG. 1 will be described in detail later.

The power transmission part 300 of the surgical instrument 10 according to one embodiment of the present disclosure may include a wire 301, a wire 302, a wire 303 (not shown), a wire 304 (not shown), a wire 305, and a wire 306, and a blade wire (not shown).

Here, the wire 301 and the wire 305 may be paired to serve as first jaw wires. The wire 302 and the wire 306 may be paired to serve as second jaw wires. Here, the components encompassing the wires 301 and 305, which are first jaw wires, and the wires 302 and 306, which are second jaw wires, may be referred to as jaw wires. In addition, the wire 303 and the wire 304 may be paired to serve as pitch wires.

In addition, the power transmission part 300 of the surgical instrument 10 according to one embodiment of the present disclosure may include coupling members coupled to respective end portions of the wires to respectively couple the wires to the pulleys. Here, each of the coupling members may have various shapes, as necessary, such as a ball shape, a tube shape, and the like.

In explaining the present disclosure, a portion close to a user side, that is, a portion close to the manipulation part 200, is described as a proximal end, and a portion far from the user side, that is, a portion close to the end tool 1100 is described as a distal end.

For example, referring to FIG. 2, a description will be given under assumption that a portion of the end tool 1100 close to the manipulation part 200 is a proximal end and a portion far from the manipulation part 200, namely, a portion close to the end portion of the end tool 1100 is a distal end of the end tool 1100. To explain this from another perspective, the proximal end of the end tool 1100 may be described as a portion close to the connection part 400, and the distal end of the end tool 1100 may be described as a portion far from the connection part 400.

Referring back to FIGS. 1 to 4, the manipulation part 200 of the surgical instrument 10 according to one embodiment of the present disclosure includes a first handle 204 that can be gripped by a user, an actuation manipulation part 203 for controlling an actuation motion of the end tool 1100, a yaw manipulation part 202 for controlling a yaw motion of the end tool 1100, and a pitch manipulation part 201 for controlling a pitch motion of the end tool 1100. Here, it can be understood that only components related to the pitch/yaw/actuation motions of the surgical instrument 10 are illustrated in FIGS. 3 and 4.

In addition, the manipulation part 200 of the surgical instrument 10 further includes a cutting manipulation part 280 that performs cutting by controlling a motion of a blade (not illustrated) of the end tool 1100, and a sealing manipulation part 270 that controls a first electrode (not illustrated) and a second electrode (not illustrated) to perform elcetrocauterization by supplying electrical energy.

The manipulation part 200 may include a pulley 211, a pulley 212, a pulley 213, a pulley 214, and a pulley 215, a pulley 217, a pulley 218, a pulley 219, and a pulley 220 related to a rotational motion of a first jaw 1101. The manipulation part 200 may also include a pulley 221, a pulley 222, a pulley 223, a pulley 224, a pulley 225, a pulley 227, a pulley 228, a pulley 229, and a pulley 230 related to a rotational motion of a second jaw 1102. In addition, the manipulation part 200 may include a pulley 262 related to the rotational motions of the first jaw 1101 and the second jaw 1102. Additionally, the manipulation part 200 may include a pulley 231 related to a pitch motion. In addition, the manipulation part 200 may include relay pulleys 235 disposed at some places along the curved part 402 of the connection part 400.

Here, the pulleys facing each other in the drawings are shown formed in parallel to each other, but are not limited thereto, and the location and size of each pulley may vary to be appropriate for the configuration of the manipulation part.

Further, the manipulation part 200 according to one embodiment of the present disclosure may include a rotation shaft 241, a rotation shaft 242, a rotation shaft 243, a rotation shaft 244, a rotation shaft 245, the rotation shaft 246, and a rotation shaft 247. Here, the rotation shaft 241 may function as a manipulation part actuation rotation shaft, the rotation shaft 247 may function as a grip pulley rotation shaft, and the rotation shaft 242 may function as a manipulation part first yaw sub-rotation shaft. In addition, the rotation shaft 243 may function as a manipulation part yaw main rotation shaft, and the rotation shaft 244 may function as a manipulation part second yaw sub-rotation shaft. Additionally, the rotation shaft 245 may function as a manipulation part pitch sub-rotation shaft, and the rotation shaft 246 may function as a manipulation part pitch main rotation shaft.

The rotation shaft 241, the rotation shaft 247, the rotation shaft 242, the rotation shaft 243, the rotation shaft 244, the rotation shaft 245, and the rotation shaft 246 may be disposed sequentially from the distal end 205 to the proximal end 206 of the manipulation part 200.

Each of the rotation shafts 241, 242, 243, 244, 245, 246, and 247 may be fitted into one or more pulleys, which will be described in detail later.

The pulley 262 may function as an actuation pulley of the first jaw and the second jaw, and may be referred to as a grip pulley.

The pulley 211 and the pulley 212 may function as manipulation part first jaw first yaw sub-pulleys, and the pulley 221 and the pulley 222 may function as manipulation part second jaw first yaw sub-pulleys. These components may also be collectively referred to as a manipulation part first yaw sub-pulley.

The pulley 213 and the pulley 214 may function as manipulation part first jaw yaw main pulleys, and the pulley 223 and the pulley 224 may function as manipulation part second jaw yaw main pulleys. These components may also be collectively referred to as a manipulation part yaw main pulley.

The pulley 215 may function as a manipulation part first jaw second yaw sub-pulley, and the pulley 225 may function as a manipulation part second jaw second yaw sub-pulley. These components may also be collectively referred to as a manipulation part second yaw sub-pulley.

The pulley 217 and the pulley 218 may function as manipulation part first jaw pitch sub-pulleys, and the pulley 227 and the pulley 228 may function as manipulation part second jaw pitch sub-pulleys. These components may also be collectively referred to as a manipulation part pitch sub-pulley.

The pulley 219 and the pulley 220 may function as manipulation part first jaw pitch main pulleys, and the pulley 229 and the pulley 230 may function as manipulation part second jaw pitch main pulleys. These components may also be collectively referred to as a manipulation part pitch main pulley.

The pulley 231 may function as a manipulation part pitch wire main pulley, and may include a pulley (not illustrated) that functions as a manipulation part pitch wire sub-pulley.

The above components are categorized from the perspective of the manipulation part for each motion (pitch/yaw/actuation) as follows.

The pitch manipulation part 201 configured to control a pitch motion of the end tool 1100 may include the pulley 217, the pulley 218, the pulley 219, the pulley 220, the pulley 227, the pulley 228, the pulley 229, the pulley 230, and the pulley 231. In addition, the pitch manipulation part 201 may include the rotation shaft 245 and the rotation shaft 246. In addition, the pitch manipulation part 201 may further include a pitch frame 208.

The yaw manipulation part 202 configured to control a yaw motion of the end tool 1100 may include the pulley 211, the pulley 212, the pulley 213, the pulley 214, the pulley 215, the pulley 221, the pulley 222, the pulley 223, the pulley 224, and the pulley 225. In addition, the yaw manipulation part 202 may include the rotation shaft 242, the rotation shaft 243, and the rotation shaft 244. In addition, the yaw manipulation part 202 may further include a yaw frame 207.

The actuation manipulation part 203 configured to control an actuation motion of the end tool 1100 may include the pulley 262, the rotation shaft 241, and the rotation shaft 247.

Hereinafter, each component of the manipulation part 200 will be described in more detail.

The first handle 204 may be formed to be gripped by a user with a hand, and in particular, may be formed to be grasped by the user by wrapping the first handle 204 with his/her palm. In addition, the actuation manipulation part 203 and the yaw manipulation part 202 are formed on the first handle 204, and the pitch manipulation part 201 is formed on one side of the yaw manipulation part 202. In addition, the other end portion of the pitch manipulation part 201 is connected to the curved part 402 of the connection part 400.

The actuation manipulation part 203 includes a grip lever 261, a grip pulley 262, and an actuation restoration elastic member (not illustrated).

Here, the grip lever 261 may be formed in the shape of a hand ring and may act as a second handle.

Here, the rotation shaft 241, which is the actuation rotation shaft, may be formed to form a predetermined angle with an XZ plane on which the connection part 400 is formed.

For example, the rotation shaft 241 may be formed in a direction parallel to the Y-axis, and in this state, when the pitch manipulation part 201 or the yaw manipulation part 202 rotates, the coordinate system of the actuation manipulation part 203 may change relatively. Of course, the concept of the present disclosure is not limited thereto, and the rotation shaft 241 may be formed in various directions so as to be suitable for a structure of the hand of the user gripping the actuation manipulation part 203 according to an ergonomic design.

Meanwhile, the actuation manipulation part 203 may further include a grip gear 261c and a pulley gear 263, and the grip lever 261, the grip gear 261c, the grip pulley 262, and the pulley gear 263 will be described in detail later.

The yaw manipulation part 202 may include the rotation shaft 242, the rotation shaft 243, the pulleys 213 and 214, which are manipulation part first jaw yaw main pulleys, the pulleys 223 and 224, which are manipulation part second jaw yaw main pulleys, and the yaw frame 207. In addition, the yaw manipulation part 202 may further include the pulleys 211 and 212, which are manipulation part first jaw first yaw sub-pulleys formed on one side of the pulleys 213 and 214, and the pulleys 221 and 222 that are manipulation part second jaw first yaw sub-pulleys formed on one side of the pulleys 223 and 224. Furthermore, the yaw manipulation part 202 may further include the pulley 215, which is a manipulation part first jaw second yaw sub-pulley formed on the other side of the pulleys 213 and 214, and the pulley 225 that is a manipulation part second jaw second yaw sub-pulley formed on the other side of the pulleys 223 and 224. Here, the pulley 215 and the pulley 225 may be coupled to the pitch frame 208, which will be described later.

Here, it is illustrated in the drawings that the yaw manipulation part 202 includes the pulleys 213 and 214 and the pulleys 223 and 224, and the pulleys 213 and 214 and the pulleys 223 and 224 are each configured as a pair of pulleys that face each other and are independently rotatable, but the concept of the present disclosure is not limited thereto. That is, one or more pulleys having the same diameter or different diameters may be disposed according to the configuration of the yaw manipulation part 202.

In detail, the rotation shaft 243, which is a manipulation part first yaw sub-rotation shaft, is formed on one side of the actuation manipulation part 203 on the first handle 204, and the rotation shaft 243, which is a manipulation part yaw main rotation shaft, is formed on one side of the rotation shaft 242. At this time, the first handle 204 is formed to be rotatable around the rotation shaft 243.

Here, the rotation shaft 243 may be formed to form a predetermined angle with the XY plane on which the connection part 400 is formed. For example, the rotation shaft 243 may be formed in a direction parallel to the Z-axis, and in this state, when the pitch manipulation part 201 rotates, the coordinate system of the rotation shaft 243 may change relatively as described above. Of course, the concept of the present disclosure is not limited thereto, and the rotation shaft 243 may be formed in various directions so as to be suitable for a structure of the hand of the user gripping the manipulation part 200 according to an ergonomic design.

Meanwhile, the pulleys 213 and 214 and the pulleys 223 and 224 are coupled to the rotation shaft 243 so as to be rotatable around the rotation shaft 243. In addition, the wire 301 or the wire 305, which is a first jaw wire, is wound around the pulleys 213 and 214, and the wire 302 or the wire 306, which is a second jaw wire, may be wound around the pulleys 223 and 224. In this case, the pulleys 213 and 214 and the pulleys 223 and 224 may each be configured as a pair of pulleys that face each other and are independently rotatable. Accordingly, a wire being wound and a wire being unwound may be wound around separate pulleys, respectively, so that the wires can perform motions without interference with each other.

The yaw frame 207 rigidly connects the first handle 204, the rotation shaft 242, and the rotation shaft 243, and the actuation manipulation part 203, which includes the rotation shaft 241 and the rotation shaft 247 and to which the grip pulley is coupled, is rigidly connected to the yaw frame 207 directly or through a relay member, so that the first handle 204, the yaw manipulation part 202, and the actuation manipulation part 203 are integrally yaw-rotated around the rotation shaft 243.

The pitch manipulation part 201 may include the rotation shaft 246, the pulleys 217 and 218, which are manipulation part first jaw pitch main pulleys, the pulleys 229 and 230, which are manipulation part second jaw pitch main pulleys, and the pitch frame 208. In addition, the pitch manipulation part 201 may further include the rotation shaft 245, the pulleys 217 and 218, which are manipulation part first jaw pitch sub-pulleys formed on one side of the pulley 219 and the pulley 220, and the pulleys 227 and 228, which are manipulation part second jaw pitch sub-pulleys formed on one side of the pulley 229 and the pulley 230. The pitch manipulation part 201 may be connected to the curved part 402 of the connection part 400 through the rotation shaft 246.

In detail, the pitch frame 208 is a base frame of the pitch manipulation part 201, and the rotation shaft 243 is rotatably coupled to one end portion of the pitch frame 208. That is, the yaw frame 207 is formed to be rotatable around the rotation shaft 243 with respect to the pitch frame 208.

As described above, since the yaw frame 207 connects the first handle 204, the rotation shaft 243, the rotation shaft 242, the rotation shaft 247, and the rotation shaft 241, and the yaw frame 207 is also axially coupled to the pitch frame 208, when the pitch frame 208 is pitch-rotated around the rotation shaft 246, the yaw frame 207 connected to the pitch frame 208, the first handle 204, the rotation shaft 241, the rotation shaft 247, the rotation shaft 242, and the rotation shaft 243 are pitch-rotated together. That is, when the pitch manipulation part 201 is rotated around the rotation shaft 246, the actuation manipulation part 203 and the yaw manipulation part 202 are rotated together with the pitch manipulation part 201. In other words, when the user pitch-rotates the first handle 204 around the rotation shaft 246, the actuation manipulation part 203, the yaw manipulation part 202, and the pitch manipulation part 201 are moved together.

The pulleys 219 and 220 and the pulleys 229 and 230 are coupled to the rotation shaft 246 so as to be rotatable around the rotation shaft 246 of the pitch frame 208.

Here, the pulley 219 and the pulley 220 may be formed to face each other so as to be independently rotatable. Accordingly, a wire being wound and a wire being unwound may be wound around separate pulleys, respectively, so that the wires can perform motions without interference with each other. Likewise, the pulley 229 and the pulley 230 may also be formed to face each other so as to be independently rotatable. Accordingly, a wire being wound and a wire being unwound may be wound around separate pulleys, respectively, so that the wires can perform motions without interference with each other.

A connection relationship of each of the first handle 204, the pitch manipulation part 201, the yaw manipulation part 202, and the actuation manipulation part 203 is summarized as follows. The rotation shafts 241, the rotation shaft 242, the rotation shaft 243, the rotation shaft 244, the rotation shaft 245, the rotation shaft 246, and the rotation shaft 247 may be formed on the first handle 204. In this case, since the rotation shafts 242 and the rotation shaft 243 are directly formed on the first handle 204, the first handle 204 and the yaw manipulation part 202 may be directly connected to each other. On the other hand, since the pitch manipulation part 201 is formed on one side of the yaw manipulation part 202 to be connected to the yaw manipulation part 202, the pitch manipulation part 201 is not directly connected to the first handle 204, and the pitch manipulation part 201 and the first handle 204 may be formed to be indirectly connected through the yaw manipulation part 202. Furthermore, since the actuation manipulation part 203 is formed on the other side of the yaw manipulation part 202 to be connected to the yaw manipulation part 202, the actuation manipulation part 203 may not be directly connected to the first handle 204, but be indirectly connected to the first handle 204 through the yaw manipulation part 202.

Continuing to refer to the drawings, in the surgical instrument 10 according to one embodiment of the present disclosure, the pitch manipulation part 201 and the end tool 1100 may be formed on the same or parallel axis (X-axis). That is, the rotation shaft 246 of the pitch manipulation part 201 is formed at one end portion of the curved part 402 of the connection part 400, and the end tool 1100 is formed at the other end portion of the connection part 400.

In addition, one or more relay pulleys 235 configured to change or guide paths of the wires may be disposed at some places along the connection part 400, particularly in the curved part 402. As at least some of the wires are wound around the relay pulleys 235 to guide the paths of the wires, these wires may be disposed along a curved shape of the curved part 402.

Here, in the drawings, it is illustrated that the connection part 400 is formed to be curved with a predetermined curvature by having the curved part 402, but the concept of the present disclosure is not limited thereto, and the connection part 400 may be formed linearly or to be curved one or more times as necessary, and even in this case, it may be said that the pitch manipulation part 201 and the end tool 1100 are formed on substantially the same axis or parallel axes. In addition, although FIG. 2 illustrates that each of the pitch manipulation part 201 and the end tool 1100 is formed on an axis parallel to the X-axis, the concept of the present disclosure is not limited thereto, and the pitch manipulation part 201 and the end tool 1100 may be formed on different axes.

Hereinafter, actuation, yaw, and pitch motions in the embodiment of the present disclosure will be described.

First, the actuation motion will be described below.

When a user rotates the grip lever 261 using his or her finger while inserting the finger into a hand ring formed on the grip lever 261, the grip pulley 262 rotates around the rotation shaft 247 in response to the rotation of the grip lever 261.

At this time, as the grip pulley 262 rotates, the wires 301 and 305 fixedly coupled to the grip pulley 262 at one end portion thereof and the wires 302 and 306 fixedly coupled to the grip pulley 262 at one end portion thereof are also moved. Here, the wires 301, 302, 305, and 306 are coupled to the single grip pulley 262, but the movements of the wires according to the rotation of the pulley differ depending on the direction in which each wire is wound around the pulley 262. This will be explained in detail later.

In addition, a rotating force is transmitted to the end tool 1100 through the power transmission part 300, and two jaws 1103 of the end tool 1100 perform an actuation motion.

Here, as described above, the actuation motion refers to a motion in which two jaws 1101 and 1102 are splayed or closed while being rotated in opposite directions. That is, when the grip lever 261 of the actuation manipulation part 203 is rotated in a direction toward the first handle 204, the first jaw 1101 is rotated in a counterclockwise direction and the second jaw 1102 is rotated in a clockwise direction, thereby closing the end tool 1100. On the other hand, when the grip lever 261 of the actuation manipulation part 203 is rotated in a direction away from the first handle 204, the first jaw 1121 is rotated in the clockwise direction and the second jaw 1122 is rotated in the counterclockwise direction, thereby opening the end tool 1100.

Next, the yaw motion will be described below.

When a user rotates the first handle 204 around the rotation shaft 243 while gripping the first handle 204, the actuation manipulation part 203 and the yaw manipulation part 202 are yaw-rotated around the rotation shaft 243. That is, when the grip pulley 262 to which the wires 301 and 305 are fixedly coupled is rotated around the rotation shaft 243, the wires 301 and 305 wound around the pulleys 213 and 214 are moved. At this time, one of the wires 301 and 305 is wound around the pulley 213 or pulley 214, and the other of the wires 301 and 305 is unwound from the pulley 213 or pulley 214. Likewise, since the wire 302 and the wire 306 are also fixedly coupled to the grip pulley 262, when the grip pulley 262 is rotated around the rotation shaft 243, the wire 302 and the wire 306 wound around the pulley 223 and the pulley 224 are moved. At this time, one of the wires 302 and 306 is wound around the pulley 223 or pulley 224, and the other of the wires 302 and 306 is unwound from the pulley 223 or pulley 224. At this time, the wire 301 and the wire 305 connected to the first jaw 1101 and the wire 302 and the wire 306 connected to the second jaw 1102 are wound around the pulleys 213 and 214 and the pulleys 223 and 224, such that the first jaw 1101 and the second jaw 1102 are rotated in the same direction during yaw rotation. In addition, a rotating force is transmitted to the end tool 1100 through the power transmission part 300, and thus a yaw motion in which the two jaws 1103 of the end tool 1100 are rotated in the same direction is performed.

At this time, since the yaw frame 207 connects the first handle 204, the rotation shaft 241, the rotation shaft 242, and the rotation shaft 243, the first handle 204, the yaw manipulation part 202, and the actuation manipulation part 203 are rotated together around the rotation shaft 243.

Next, the pitch motion will be described below.

When a user rotates the first handle 204 around the rotation shaft 246 while gripping the first handle 204, the actuation manipulation part 203, the yaw manipulation part 202, and the pitch manipulation part 201 are pitch-rotated around the rotation shaft 246. That is, when the grip pulley 262 to which the wires 301 and 305 are fixedly coupled is rotated around the rotation shaft 246, the wires 301 and 305 wound around the pulleys 219 and 220 are moved. Likewise, when the grip pulley 262 to which the wires 302 and 306 are fixedly coupled is rotated around the rotation shaft 246, the wires 302 and 306 wound around the pulleys 229 and 230 are moved. At this time, as described with reference to FIG. 5, etc., the wires 301 and 305, which are first jaw wires, are moved in the same direction and the wire 302 and the wire 306, which are second jaw wires, are moved in the same direction. Accordingly, the wire 301, the wire 305, the wire 302, and the wire 306, which are the jaw wires, are wound around the pulley 219, the pulley 220, the pulley 229, and the pulley 230, which are the manipulation part pitch main pulleys, respectively, thereby pitch-rotating the first jaw 1101 and the second jaw 1102. In addition, a rotating force is transmitted to the end tool 1100 through the power transmission part 300, and the two jaws 1103 of the end tool 1100 perform a pitch motion.

At this time, since the pitch frame 208 is connected to the yaw frame 207, and the yaw frame 207 connects the first handle 204, the rotation shaft 241, the rotation shaft 242, and the rotation shaft 243, when the pitch frame 208 is rotated around the rotation shaft 246, the yaw frame 207 connected to the pitch frame 208, the first handle 204, the rotation shaft 241, the rotation shaft 242, and the rotation shaft 243 are rotated together. That is, when the pitch manipulation part 201 is rotated around the rotation shaft 246, the actuation manipulation part 203 and the yaw manipulation part 202 are rotated together with the pitch manipulation part 201.

In summary, in the surgical instrument 10 according to one embodiment of the present disclosure, the pulleys are formed on respective joint points (an actuation joint, a yaw joint, and a pitch joint), the wires (the first jaw wire or the second jaw wire) are wound around the pulleys, and the rotational manipulations (actuation rotation, yaw rotation, and pitch rotation) of the manipulation part cause the movement of each wire, which in turn induces the desired motion of the end tool 1100. Furthermore, the auxiliary pulley may be formed on one side of each of the pulleys, and the wire may not be wound several times around one pulley due to the auxiliary pulley.

FIG. 5 is a view schematically illustrating only a configuration of pulleys and wires constituting joints of the surgical instrument 10 according to one embodiment of the present disclosure illustrated in FIG. 1. In FIG. 5, relay pulleys for changing paths of the wires regardless of the operation of joints are omitted.

Referring to FIG. 5, the manipulation part 200 may include a pulley 211, the pulley 212, the pulley 213, the pulley 214, the pulley 215, the pulley 217, the pulley 218, the pulley 219, and the pulley 220 related to a rotational motion of the first jaw 1101.

In addition, the manipulation part 200 may include the pulley 221, the pulley 222, the pulley 223, the pulley 224, the pulley 225, the pulley 227, the pulley 228, the pulley 229, and the pulley 230 related to a rotational motion of the second jaw 1122. Furthermore, the manipulation part 200 may include the pulley 262 related to the rotational motion of the first jaw and the second jaw. (the disposition and structure of each of the pulleys of the manipulation part 200 are the same in principle as the disposition and structure of each of the pulleys of the end tool 1100, and thus specific designations of some reference numerals are omitted in the drawings).

The pulleys 211 and 212 and the pulleys 221 and 222 may be formed to be rotatable independently of each other around the same shaft, that is, the rotation shaft 242. In this case, the pulley 211 and the pulley 212 may each be formed as a pair of pulleys that face each other so as to be independently rotatable. Likewise, the pulleys 221 and 222 may be formed as a pair of pulleys that face each other so as to be independently rotatable, and in this case, the two pulleys may be formed to have different diameters.

The pulleys 213 and 214 and the pulleys 223 and 224 may be formed to be rotatable independently of each other around the same shaft, that is, the rotation shaft 243. In this case, the pulleys 213 and 214 and the pulleys 223 and 224 may each be configured as a pair of pulleys that face each other and are independently rotatable.

The pulley 215 and the pulley 225 may be formed to be rotatable independently of each other around the same shaft, that is, the rotation shaft 244.

The pulleys 217 and 218 and the pulleys 227 and 228 may be formed to be rotatable independently of each other around the same shaft, that is, the rotation shaft 245. At this time, the pulley 217 and the pulley 218 may be formed to have different diameters. In addition, the pulley 227 and the pulley 228 may be formed to have different diameters.

The pulleys 219 and 220 and the pulleys 229 and 230 may be formed to be rotatable independently of each other around the same shaft, that is, the rotation shaft 246.

The wire 301 is wound around the pulley 262 after sequentially passing through the pulley 219, the pulley 217, the pulley 215, the pulley 213, and the pulley 211 of the manipulation part 200, and then is coupled to the pulley 262 by a coupling member (not illustrated). Meanwhile, the wire 305 sequentially passes through the pulley 220, the pulley 218, the pulley 214, and the pulley 212 of the manipulation part 200 and is coupled to the pulley 262 by a coupling member (not illustrated). Here, the coupling member may be directly coupled to the pulley 262, or the coupling member may be coupled to a separate fastening member (not illustrated) and the fastening member may be coupled to the pulley 262, so that the coupling member is coupled to the pulley 262. Therefore, when the pulley 262 is rotated, the wire 301 and the wire 305 are wound around or unwound from the pulley 262, causing the first jaw 1101 to be rotated.

The wire 306 is wound around the pulley 262 after sequentially passing through the pulley 229, the pulley 227, the pulley 225, the pulley 223, and the pulley 221 of the manipulation part 200, and then is coupled to the pulley 262 by a coupling member (not illustrated). Meanwhile, the wire 302 sequentially passes through the pulley 230, the pulley 228, the pulley 224, and the pulley 222 of the manipulation part 200 and is coupled to the pulley 262 by a coupling member (not illustrated). Here, the coupling member may be directly coupled to the pulley 262, or the coupling member may be coupled to a separate fastening member and the fastening member may be coupled to the pulley 262, so that the coupling member is coupled to the pulley 262. Therefore, when the pulley 262 is rotated, the wire 302 and the wire 306 are wound around or unwound from the pulley 262, causing the second jaw 1102 to be rotated.

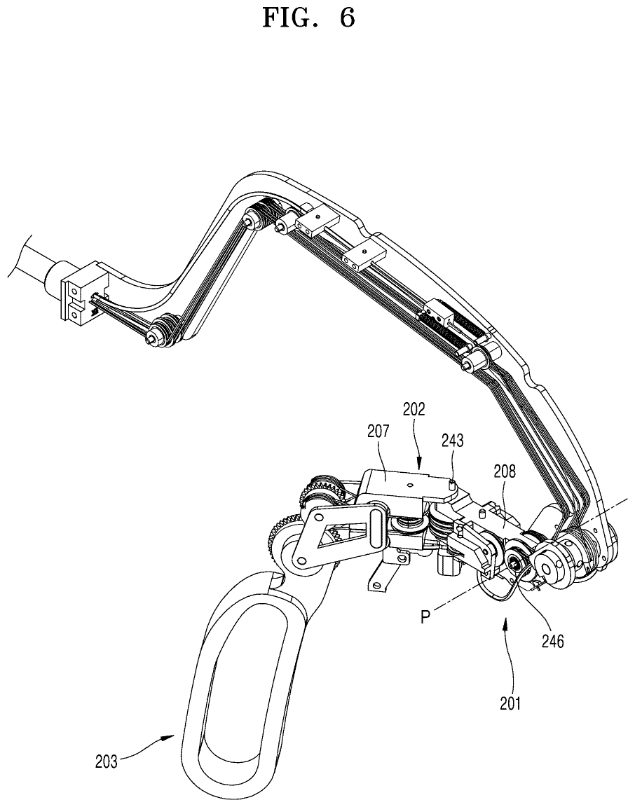

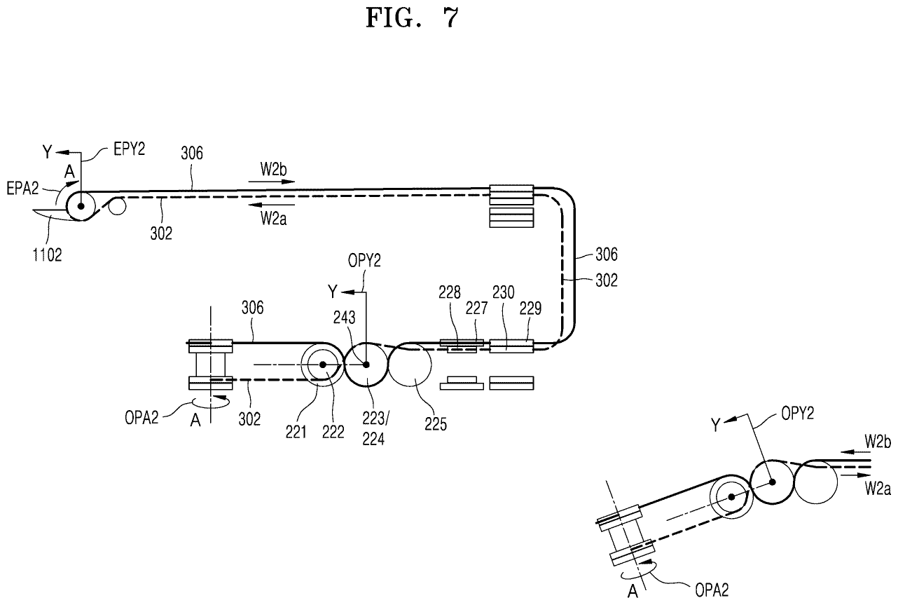

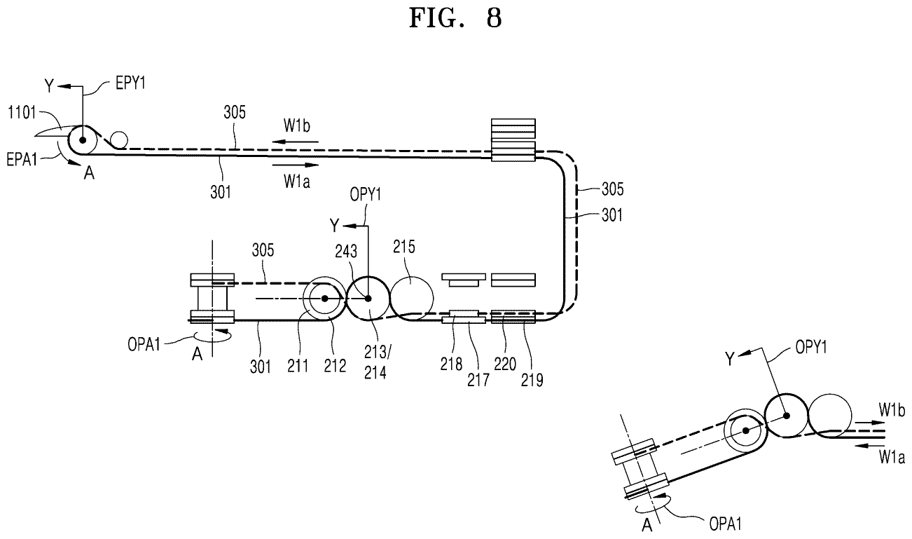

FIGS. 7 and 8 are diagrams illustrating a configuration of pulleys and wires, which are related to an actuation motion and a yaw motion of the surgical instrument 10 according to one embodiment of the present disclosure illustrated in FIG. 1, in detail for each of the first jaw and the second jaw FIG. 7 is a diagram illustrating only pulleys and wires related to the second jaw, and FIG. 8 is a diagram illustrating only pulleys and wires related to the first jaw. And, FIG. 6 is a perspective view illustrating the yaw motion of the surgical instrument of FIG. 1.

First, a wire motion of the actuation motion will be described.

Referring to FIG. 8, when the pulley gear 263 is rotated around the rotation shaft 247 in a direction of an arrow OPA1 in response to the rotation of the grip gear 261c of the grip lever 261 around the rotation shaft 241, the grip pulley 262 coupled to the pulley gear 263 is rotated in the direction of the arrow OPA1, and the wire 301 and the wire 305 wound around the grip pulley 262 are moved in directions W1a and W1b, respectively, such that the first jaw 1101 of the end tool 1100 is rotated in a direction of an arrow EPA1.

Referring to FIG. 7, when the pulley gear 263 is rotated around the rotation shaft 247 in a direction of an arrow OPA2 in response to the rotation of the grip gear 261c of the grip lever 261 around the rotation shaft 241, the grip pulley 262 connected to the pulley gear 263 is rotated in the direction of an arrow OPA2, and thus both strands of the wire 302 and wire 306 wound around the grip pulley 262 are moved in directions W2a and W2b, respectively, such that the second jaw 1102 of the end tool 1100 is rotated in a direction of an arrow EPA2. Accordingly, when a user manipulates the grip lever 261 in a direction close to each other, a motion of the first jaw 1101 and the second jaw 1102 of the end tool being close to each other is performed.

Next, a wire motion of the yaw motion will be described.

First, since the rotation shaft 243 is connected to the rotation shafts 241 and 242 by the yaw frame (see 207 of FIG. 3), the rotation shaft 243 and the rotation shafts 241 and 242 are integrally rotated together.

Referring to FIG. 8, when the first handle 204 is rotated around the rotation shaft 243 in a direction of an arrow OPY1, the pulley 262, the pulley 211, the pulley 212, the pulley 213, and the pulley 214 and the wires 301 and 305 wound therearound are rotated as a whole around the rotation shaft 243. As a result, the wires 301 and 305 wound around the pulleys 213 and 214 are moved in the directions W1a and W1b, respectively, which in turn causes the first jaw 1101 of the end tool 1100 to rotate in a direction of an arrow EPY1.

Referring to FIG. 7, when the first handle 204 is rotated around the rotation shaft 243 in a direction of an arrow OPY2, the pulley 262, the pulley 221, the pulley 222, the pulley 223, and the pulley 224 and the wires 302 and 306 wound therearound are rotated as a whole around the rotation shaft 243. As a result, the wires 302 and 306 wound around the pulleys 223 and 224 are moved in a direction opposite to the direction W2a and a direction opposite to the direction W2b, respectively, which in turn causes the first jaw 1101 of the end tool 1100 to rotate in a direction of an arrow EPY2.

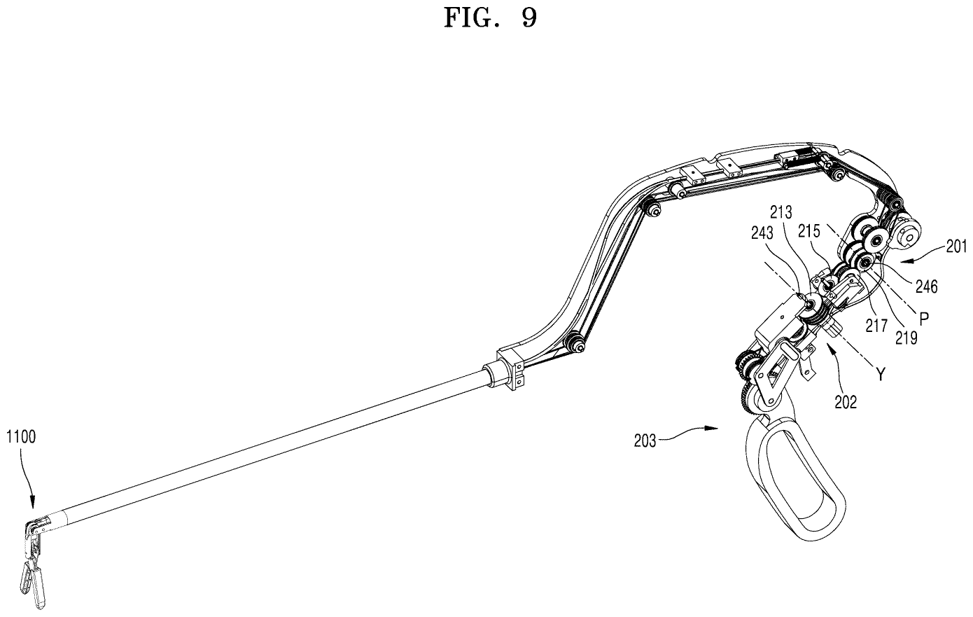

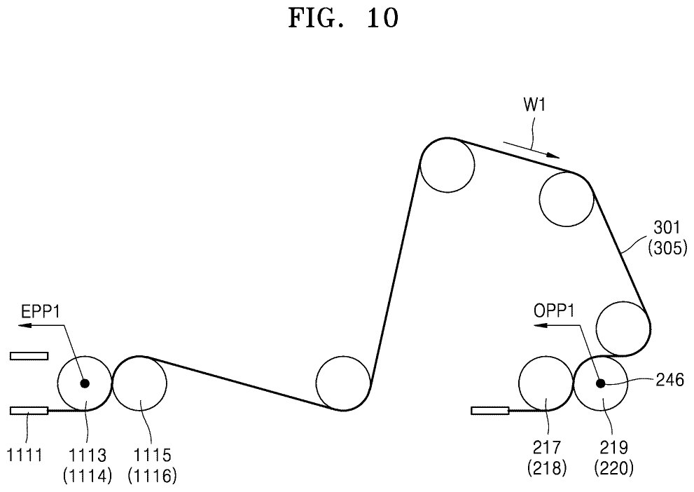

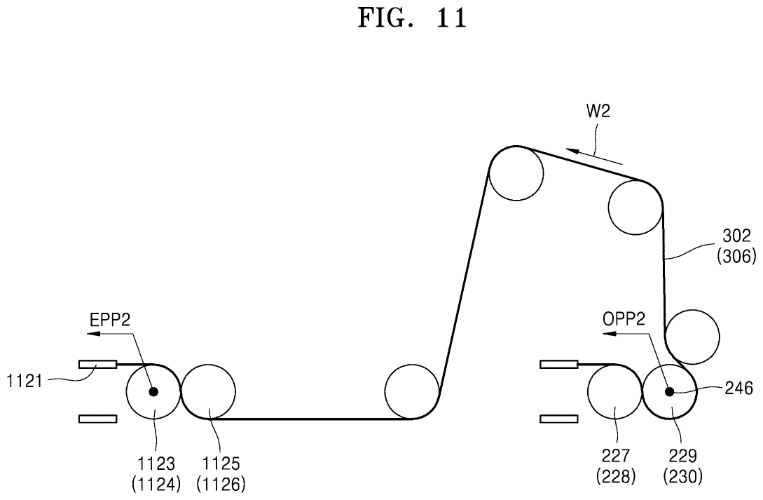

FIGS. 10 and 11 are diagrams illustrating a configuration of pulleys and wires, which are related to a pitch motion of the surgical instrument 10 according to one embodiment of the present disclosure illustrated in FIG. 1, in detail for each of the first jaw and the second jaw FIG. 11 is a diagram illustrating only pulleys and wires related to the second jaw, and FIG. 10 is a diagram illustrating only pulleys and wires related to the first jaw. As illustrated in FIG. 1 and elsewhere herein, there are two pulleys related to the pitch motion, and both strands of each wire are wound in the same path, which is illustrated with one line in FIG. 10. And, FIG. 9 is a perspective view illustrating a pitch motion of the surgical instrument of FIG. 1.

Referring to FIG. 10, when the first handle 204 is rotated around the rotation shaft 246 in a direction of an arrow OPP1, the pulley 262, the pulley 217, the pulley 219, and the like, and the wire 301 and the like wound therearound are rotated as a whole around the rotation shaft 246. At this time, since the wires 301 and 305, which are first jaw wires, are wound around upper portions of the pulley 219 and the pulley 220, the wires 301 and 305 are moved in a direction of an arrow W1. Accordingly, the first jaw 1101 of the end tool 1100 is rotated in a direction of an arrow EPP1.

Referring to FIG. 11, when the first handle 204 is rotated around the rotation shaft 246 in a direction of an arrow OPP2, the pulley 262, the pulley 227, the pulley 229, and the like, and the wire 302 and the like wound therearound are rotated as a whole around the rotation shaft 246. At this time, since the wires 302 and 306, which are second jaw wires, are wound around lower portions of the pulley 229 and the pulley 230, the wires 302 and 306 are moved in a direction of an arrow W2. Accordingly, the second jaw 1102 of the end tool 1100 is rotated in a direction of an arrow EPP2.

Thus, the actuation, yaw, and pitch manipulations are independently manipulatable.

Hereinafter, the actuation manipulation part 203 will be described in detail.

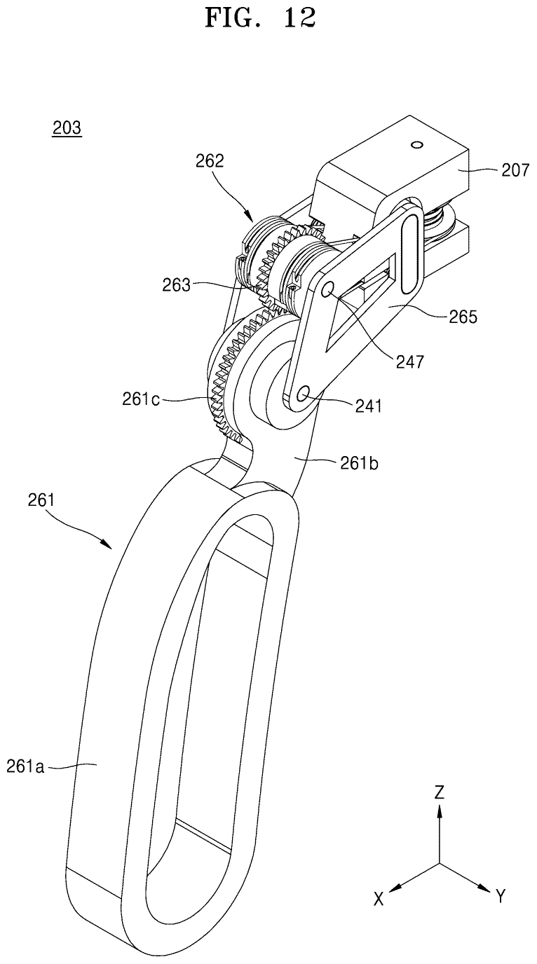

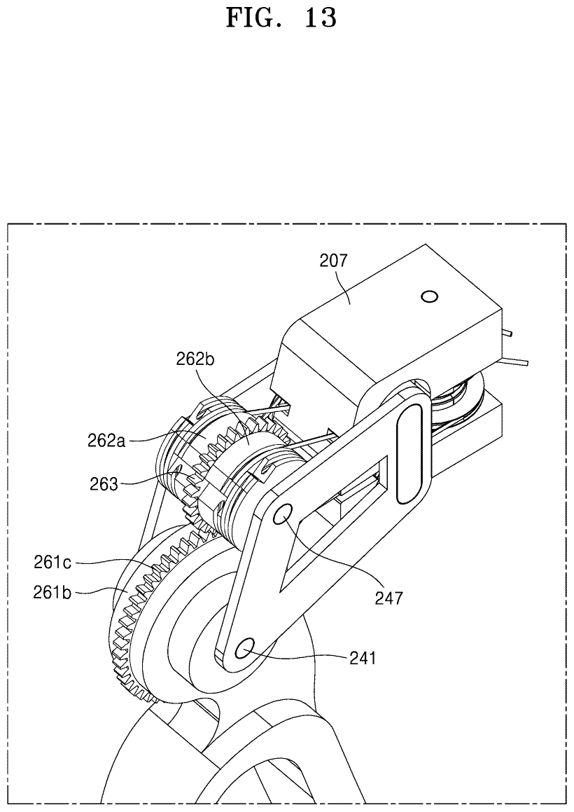

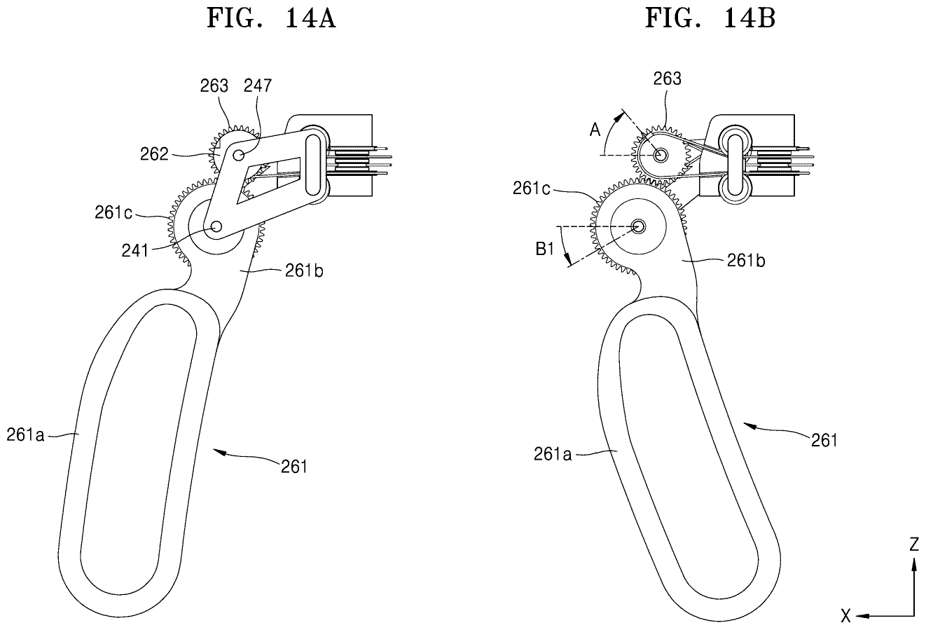

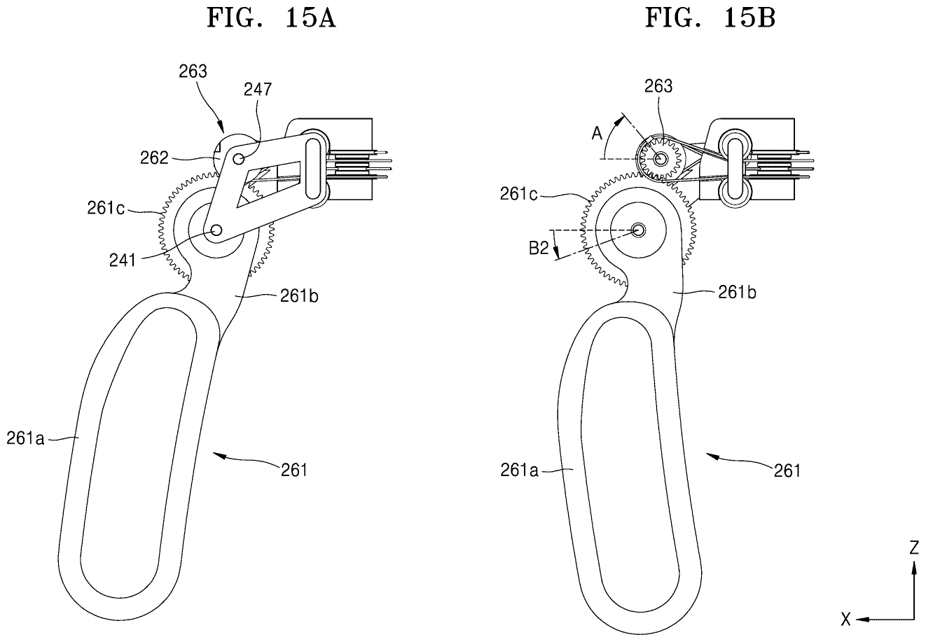

FIG. 12 is a perspective view illustrating a manipulation part of a surgical instrument according to one embodiment of the present disclosure, and FIG. 13 is a perspective view illustrating the manipulation part of the surgical instrument of FIG. 12 from a different angle. FIGS. 14A and 14B are views illustrating an actuation motion of the manipulation part of the surgical instrument of FIG. 12. FIGS. 15A and 15B are views illustrating a manipulation part of a surgical instrument according to a modified example of the one embodiment of the present disclosure, which illustrates an actuation motion of the manipulation part.

The manipulation part 200 of the surgical instrument according to one embodiment of the present disclosure, as described above, may include the handle and the actuation manipulation part 203.

In detail, the actuation manipulation part 203 may include the grip lever 261, the grip gear 261c, the pulley gear 263, and the grip pulley 262.

The grip lever 261 may be disposed adjacent to the handle and may be rotatable around the actuation rotation shaft 241. To explain this from another perspective, the grip lever 261 may be disposed adjacent to the yaw frame 207 and may be connected to the yaw frame 207 through the actuation rotation shaft 241. A user may manipulate the grip lever 261 to cause the actuation motion of the end tool 1100.

In detail, the grip lever 261 may include a grip part 261a, a rotating part 261b, and a grip gear 261c.

Here, the grip part 261a may be formed in the shape of a hand ring and may operate as a second handle.

The grip part 261a may extend from the rotating part 261b and may be a portion that a user grips to transmit force to the grip lever 261.

In other words, the rotating part 261b may be formed on one end portion of the grip part 261a. Here, the rotating part 261b is a part into which the actuation rotation shaft 241 is inserted and may be formed to be rotatable around the actuation rotation shaft 241.

The rotating part 261b may be formed in a disk shape with a certain diameter based on the actuation rotation shaft 241. Additionally, the rotating part 261b may be a part on which the grip gear 261c, which will be described later, is formed. The rotating part 261b may be formed integrally with the grip part 261a, but may alternatively be a member that is formed as a separate member and coupled to the grip part 261a.

Meanwhile, the rotating part 261b may be connected to an actuation frame 265 through the actuation rotation shaft 241.

Here, the actuation frame 265 is connected to the yaw frame 207 and may be a member through which the grip pulley rotation shaft 247 and the actuation rotation shaft 241 are axially coupled to each other. That is, the grip pulley rotation shaft 247 and the actuation rotation shaft 241 may be coupled to the actuation frame 265 so as to be connected to the yaw frame 207 of the manipulation part 200. Also, the grip lever 261 may be coupled to the actuation frame 265 through the actuation rotation shaft 241, and the grip pulley 262 may be coupled to the actuation frame 265 through the grip pulley rotation shaft 247. That is, the grip lever 261 and the grip pulley 262 may be connected to the yaw frame 207 through the actuation frame 265.

The actuation frame 265 may be coupled to one end portion of the actuation rotation shaft 241 and one end portion of the grip pulley rotation shaft 247. Additionally, the actuation frame 265 may be provided as a pair of frames facing each other on both sides with respect to the grip pulley 262.

That is, at least an area of the grip pulley 262 may be disposed between the pair of actuation frames 265.

Likewise, at least an area of the rotating part 261b may be disposed between the pair of actuation frames 265.

Meanwhile, the actuation rotation shaft 241 may be a rotation shaft parallel to the grip pulley rotation shaft 247. For example, referring to FIG. 12, both the actuation rotation shaft 241 and the grip pulley rotation shaft 247 may be formed to be parallel to the Y-axis.

The manipulation part 200 of the surgical instrument according to one embodiment of the present disclosure is configured to be actuated in a manner that the grip lever 261 and the grip pulley 262 are coupled to different rotation shafts. In this way, the actuation rotation shaft 241, around which the grip lever 261 rotates, may be formed as a different rotation shaft from the grip pulley rotation shaft 247, around which the grip pulley 262 rotates, resulting in securing freedom in design. In other words, according to an embodiment of the present disclosure, one or more degrees of freedom can be added in terms of design compared to a structure in which the rotation shaft of the grip lever 261 and the rotation shaft of the grip pulley 262 are the same as each other.

For example, rotating force and rotation angle of the grip lever 261 may be changed by variously changing the diameter of the grip pulley 262 and the diameter of the rotating part 261b of the grip lever 261. This will be explained later.

Meanwhile, the grip gear 261c may rotate around the actuation rotation shaft 241, in response to the rotation of the grip lever 261. In addition, the grip gear 261c may be fixedly coupled to the rotating part 261b and rotate together, or may be formed integrally with the rotating part 261b.

Specifically, the grip gear 261c may be formed along the circumference of the rotating part 261b. For example, the grip gear 261c may be formed along the circumference of the rotating part 261b excluding a portion, which extends from one side of the rotating part 261b, namely, a portion where the grip part 261a is formed. In addition, the diameter of the grip gear 261c may be larger than the diameter of the rotating part 261b.

Here, the grip gear 261c may be a gear that is rotatable in engagement with the pulley gear 263, which will be described later.

Therefore, when the grip lever 261 rotates around the actuation rotation shaft 241, the grip gear 261c also rotates around the same shaft, so the pulley gear 263 engaged with the grip gear 261c can rotate.

As described above, the pulley gear 263 may be a gear engaged with the grip gear 261c. And, the pulley gear 263 may rotate around the grip pulley rotation shaft 247. That is, the rotation shaft of the pulley gear 263 may be the same rotation shaft as that of the grip pulley 262. Additionally, the pulley gear 263 may be coupled to one side surface of the grip pulley 262 and rotate integrally with the grip pulley 262.

As an example, the grip pulley 262 may include a first grip pulley 262a coupled to one side with respect to the pulley gear 263, and a second grip pulley 262b coupled to the other side with respect to the pulley gear 263. The first grip pulley 262a, the pulley gear 263, and the second grip pulley 262b may be integrally coupled together so as to rotate around the same rotation shaft.

In other words, the pulley gear 263 may be a gear that is interposed between the first grip pulley 262a and the second grip pulley 262b and integrally coupled with the first grip pulley 262a and the second grip pulley 262b so as to rotate together with the grip pulley 262.

As another embodiment, the diameter of the pulley gear 263 may be smaller than the diameter of the grip pulley 262. Specifically, the tip diameter of the pulley gear 263 may be smaller than the diameter of the grip pulley 262. In this case, since the pulley gear 263 is interposed between the first grip pulley 262a and the second grip pulley 262b, the teeth of the pulley gear 263 may not protrude out of the outer circumferential surface of the grip pulley 262. Therefore, in order for the grip gear 261c to engage with the pulley gear 263, at least a portion of the grip gear 261c may be interposed between the first grip gear 262a and the second grip gear 262b.

Thus, when the pulley gear 263 engaged with the grip gear 261c rotates, the grip pulley 262 may rotate around the grip pulley rotation shaft 247, in response to the rotation of the pulley gear 263. That is, as the grip lever 261 rotates, the grip gear 261c may rotate and in turn the pulley gear 263 engaged with the grip gear 261c may rotate, causing the grip pulley 262 to rotate.

To explain this from another perspective, when the grip lever 261 rotates in a first direction, the grip gear 261c may rotate in the first direction and the pulley gear 263 and the grip pulley 262 may rotate in a second direction that is opposite to the first direction.

In the manipulation part 200 of the surgical instrument according to one embodiment of the present disclosure, when the grip lever 261 rotates, a rotating force transmitted to the grip pulley 262 and a rotation angle displacement of the grip pulley 262 may vary depending on the ratio of the diameter of the pulley gear 263 to the diameter of the grip gear 261c.

This will be explained in detail as follows.

Referring back to FIGS. 14A, 14B, 15A and 15B, the diameters of the grip gear 261c and the pulley gear 263 of the actuation manipulation part 203 may change depending on the design. For example, it can be seen that the diameters of the grip gear 261c and the pulley gear 263 of the actuation manipulation part 203 illustrated in FIGS. 15A and 15B are respectively different from the diameters of the grip gear 261c and the pulley gear 263 of the actuation manipulation part 203 illustrated in FIGS. 14A and 14B.

Specifically, a distance between the actuation rotation shaft 241 and the grip pulley rotation shaft 247 may be constant, but the diameter of the grip gear 261c may increase and the diameter of the pulley gear 263 may decrease. Alternatively, the diameter of the grip gear 261c may decrease and the diameter of the pulley gear 263 may increase.

That is, the actuation manipulation part 203 may achieve different combinations of variables for setting the rotation range and rotating force of the grip lever 261 and the grip pulley 262, by changing the grip gear 261c and the pulley gear 263 while the size of the actuation frame 265 is constantly maintained.

In addition, in another embodiment of the present disclosure, the actuation manipulation part 203 may, of course, be designed in more various combinations by changing the size of the actuation frame 265 and the distance between the actuation rotation shaft 241 and the grip pulley rotation shaft 247.

When the diameter of the grip gear 261c is D₁, the diameter of the pulley gear 263 is D₂, the rotating force of the grip lever 261 is M₁, the rotating force of the grip pulley 262 is M₂, the rotation angle displacement of the grip lever 261 is θ₁, and the rotation angle displacement of the grip pulley 262 is θ₂, the rotating force of the grip pulley 262 and the rotation angle displacement of the grip pulley 262 satisfy the following relationship.

$${M}_{2} = {M}_{1} \times \frac{{D}_{2}}{{D}_{1}}$$

$${θ}_{2} = {θ}_{1} \times \frac{{D}_{1}}{{D}_{2}}$$

According to an embodiment of the present disclosure, based on the relation, the rotation angle displacements of the grip lever 261 and the grip pulley 262 and the rotating forces of the grip lever 261 and the grip pulley 262 may be appropriately set through the combination of the grip gear 261c and the pulley gear 263.

For example, when the ratio of the diameter of the grip gear 261c to the diameter of the pulley gear 263 is 2:1, the rotating force of the grip pulley 262 may be ½ times the rotating force of the grip lever 261, and the rotation angle displacement of the grip pulley 262 may be twice the rotation angle displacement of the grip lever 261.

Referring to FIGS. 14A and 14B, the manipulation part 200 according to one embodiment of the present disclosure must rotate the grip lever 261 by an angle B1 in order to rotate the grip pulley 262 by an angle A.

Here, the rotation angle of the grip lever 261 for rotating the grip pulley 262 by the same angle may vary depending on a difference in gear ratio between the grip gear 261c and the pulley gear 263. That is, when the ratio of diameter of the grip pulley 262 to the diameter of the grip gear 261c varies, the rotation angle of the grip lever 261 for rotating the grip pulley 262 by the same angle may vary.

For example, as illustrated in FIGS. 15A and 15B, the manipulation part 200 according to a modified example of one embodiment of the present disclosure must rotate the grip lever 261 by an angle B2 in order to rotate the grip pulley 262 by an angle A.

As such, by the appropriate change of the diameters of the grip gear 261c and the pulley gear 263, the rotating force transmitted to the grip pulley 262 can be maintained and simultaneously the rotational motion range of the grip lever 261 can be reduced.

As a result, the manipulation part 200 of the surgical instrument according to one embodiment of the present disclosure can respond more effectively to optimization of the power transmission structure design of a multi-joint surgical instrument, which employs a wire-pulley structure by securing design flexibility as described above.

Meanwhile, the grip gear 261c and the pulley gear 263 may be directly engaged with each other, but the concept of the present disclosure is not necessarily limited thereto, and a separate auxiliary gear and a member of performing an additional function may further be disposed between the grip gear 261c and the pulley gear 263.

For example, in another embodiment, an auxiliary gear may be added between the grip gear 261c and the pulley gear 263 so that the rotation direction of the grip gear 261c matches the rotation direction of the pulley gear 263. Alternatively, the design may be made to change an angle between the actuation rotation shaft 241 and the grip pulley rotation shaft 247. In this way, according to embodiments of the present disclosure, the rotation shaft of the grip lever 261 and the rotation shaft of the grip pulley 262 can be separately employed, thereby securing design flexibility and expandability, compared to a structure having a unified shaft.

Meanwhile, one or more wires may be connected to the grip pulley 262. The grip pulley 262 may rotate around the grip pulley rotation shaft 247 so as to transmit force to the end tool 1100 through the wires connected to the grip pulley 262.

Specifically, one or more wires may be connected to each of the first grip pulley 262a and the second grip pulley 262b. Also, as the pulley gear 263 and the grip pulley 262 rotate, the wires connected to the grip pulley 262 may be wound around or unwound from the grip pulley 262.

Hereinafter, the structure for transmitting power to the end tool 1100 through the grip pulley 262 and the wires will be described in detail.

The surgical instrument according to one embodiment of the present disclosure may be actuated in such a manner that the first handle is gripped with a palm and the grip lever 261 is pulled toward the first handle 204 with a finger inserted into the grip lever 261. Accordingly, the grip gear may be rotated and the pulley gear and the grip pulley 262 may be rotated in response to the rotation of the grip gear. That is, in one embodiment of the present disclosure, an actuation motion may be performed by manipulating one lever.

As an example, referring to FIG. 5, and elsewhere herein, since the wires 301 and 305, which are first jaw wires, and the wires 302 and 306, which are second jaw wires, are all coupled to the single grip pulley 262, each wire may be appropriately disposed. This may result in varying the movement of each wire merely by the rotation of the single pulley. That is, the first jaw 1101 and the second jaw 1102 may be rotated in different directions through the rotation of the grip pulley 262 by the grip lever 261 in one direction. In other words, in response to the rotation of the grip pulley 262, the first jaw 1101 and the second jaw 1102 may perform an opening or closing motion to be open or closed.

Specifically, the wire 301 and wire 305, which are the first jaw wires, may be wound around the grip pulley 262 in opposite directions. For example, as illustrated in FIG. 5 and elsewhere herein, the wire 301 may be wound clockwise around the grip pulley 262, and the wire 305 may be wound counterclockwise around the grip pulley 262. Likewise, the wire 302 and the wire 306, which are the second jaw wires, may be wound around the grip pulley 262 in opposite directions. For example, as illustrated in FIG. 5, the wire 302 may be wound counterclockwise around grip pulley 262 and the wire 306 may be wound clockwise around the grip pulley 262.

At this time, when the grip pulley 262 rotates clockwise, the wire 301 is wound around the grip pulley 262 and the wire 305 is unwound. Accordingly, the wire 301 is unwound from and the wire 305 is wound around a first jaw pulley 1111 of the end tool 1100, so that the end tool first jaw pulley 1111 rotates counterclockwise.

In addition, when the grip pulley 262 rotates clockwise, the wire 306 is wound around the grip pulley 262 and the wire 302 is unwound. Accordingly, the wire 306 is unwound from and the wire 302 is wound around a second jaw pulley 1121 of the end tool 1100, so that the end tool second jaw pulley 1121 rotates clockwise.

Likewise, when the grip pulley 262 rotates counterclockwise, the first jaw pulley 1111 rotates clockwise, and the second jaw pulley 1121 rotates counterclockwise.

Therefore, when the grip pulley 262 rotates, the first jaw pulley 1111 and the second jaw pulley 1121 rotate in opposite directions, such that the first and second jaws of the end tool 1100 are open or closed.

So far, the present disclosure has been described with a focus on the preferred embodiment. It will be understood by a person skilled in the art to which the present disclosure pertains that the present disclosure can be implemented in modified forms without departing from the essential features of the present disclosure. Therefore, the embodiments disclosed herein should be regarded as illustrative rather than restrictive. The scope of the present disclosure is defined in the following claims rather than the foregoing description, and all differences within the equivalents should be construed as being included in the disclosure.

According to the present disclosure, design flexibility and expandability can be secured for a wire-pulley structure of a manipulation part of a surgical instrument.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. A manipulation part of a surgical instrument, the manipulation part comprising:
a handle; and
an actuation manipulation part that is disposed on one side of the handle, and controls an actuation motion of an end tool including at least one jaw,
wherein the actuation manipulation part comprises:
a grip lever that rotates around an actuation rotation shaft;
a grip gear that rotates around the actuation rotation shaft in response to rotation of the grip lever;
a pulley gear that is engaged with the grip gear; and
a grip pulley that rotates around a grip pulley rotation shaft, in response to rotation of the pulley gear, and has at least one wire connected thereto, and
the grip pulley transmits power to the end tool through the at least one wire connected to the grip pulley by the rotation around the grip pulley rotation shaft.

2. The manipulation part of claim 1, wherein
the actuation rotation shaft is parallel to the grip pulley rotation shaft.

3. The manipulation part of claim 1, wherein the grip lever comprises:
a rotating part to which the actuation rotation shaft is coupled and which is formed in a disk shape having a certain diameter based on the actuation rotation shaft; and
a grip part that is a part extending from the rotating part and gripped by a user to transmit force to the grip lever.

4. The manipulation part of claim 3,
wherein the grip gear is formed along a circumference of the rotating part.

5. The manipulation part of claim 1,
wherein the pulley gear is coupled to one side surface of the grip pulley to rotate integrally with the grip pulley.

6. The manipulation part of claim 1,
wherein the grip pulley comprises:
a first grip pulley coupled to one side with respect to the pulley gear, and a second grip pulley coupled to another side with respect to the pulley gear; and
the first grip pulley, the pulley gear, and the second grip pulley are integrally coupled together, so as to rotate around a same rotation shaft.

7. The manipulation part of claim 1,
wherein when the grip lever rotates in a first direction, the grip gear rotates in the first direction, and the pulley gear and the grip pulley rotate in a second direction opposite to the first direction.

8. The manipulation part of claim 1,
wherein when the grip lever rotates, a rotating force transmitted to the grip pulley and a rotation angle displacement of the grip pulley vary depending on a ratio of a diameter of the pulley gear to a diameter of the grip gear.

9. A surgical instrument comprising: an end tool that comprises a first jaw and a second jaw each formed to be rotatable, and is rotatable in at least two directions;
a manipulation part that controls rotation of the end tool in the at least two directions;
a power transmission part that comprises a first jaw wire connected to the manipulation part to transmit rotation of the manipulation part to the first jaw, and a second jaw wire connected to the manipulation part to transmit the rotation of the manipulation part to the second jaw; and
a connection part that has one end portion coupled to the end tool and another end portion coupled to the manipulation part, such that the manipulation part and the end tool are connected to each other,
wherein the manipulation part comprises:
a handle; and
an actuation manipulation part that is disposed on one side of the handle, and controls an actuation motion of the end tool comprising the first jaw and the second jaw,
the actuation manipulation part comprises:
a grip lever that rotates around an actuation rotation shaft;
a grip gear that rotates around the actuation rotation shaft in response to rotation of the grip lever;
a pulley gear that is engaged with the grip gear; and
a grip pulley that rotates around a grip pulley rotation shaft, in response to rotation of the pulley gear, and has at least one wire connected thereto, and
the grip pulley transmits power to the end tool through the at least one wire connected to the grip pulley by the rotation around the grip pulley rotation shaft.

10. The surgical instrument of claim 9,
wherein the actuation rotation shaft is parallel to the grip pulley rotation shaft.

11. The surgical instrument of claim 9,
wherein the grip lever comprises:
a rotating part to which the actuation rotation shaft is coupled and which is formed in a disk shape having a certain diameter based on the actuation rotation shaft; and
a grip part that is a part extending from the rotating part and gripped by a user to transmit force to the grip lever.

12. The surgical instrument of claim 9,
wherein the pulley gear is coupled to one side surface of the grip pulley to rotate integrally with the grip pulley.

13. The surgical instrument of claim 9,
wherein the grip pulley comprises
a first grip pulley coupled to one side with respect to the pulley gear, and a second grip pulley coupled to another side with respect to the pulley gear, and
the first grip pulley, the pulley gear, and the second grip pulley are integrally coupled together, so as to rotate around a same rotation shaft.

14. The surgical instrument of claim 13,
wherein the first jaw wire comprises a first strand and a second strand as a pair of wires,
the second jaw wire comprises a third strand and a fourth strand as a pair of wires,
the first strand of the first jaw wire and the fourth strand of the second jaw wire are connected to any one of the first grip pulley or the second grip pulley, and
the second strand of the first jaw wire and the third strand of the second jaw wire are connected to another one of the first grip pulley or the second grip pulley.

15. The surgical instrument of claim 9,
wherein the grip pulley is rotated by the grip lever in any one direction to enable an opening or closing motion of the first jaw and the second jaw.

16. The surgical instrument of claim 15,
wherein at least a portion of the first jaw wire and at least a portion of the second jaw wire are fixedly wound around the grip pulley.

17. The surgical instrument of claim 16,
wherein the first jaw wire and the second jaw wire each comprise a pair of wires, one of the pair of wires being wound around the grip pulley in a first direction while another one is wound around the grip pulley in a second direction opposite to the first direction.

18. The surgical instrument of claim 17,
wherein when the grip lever rotates in the first direction, the grip gear rotates in the first direction, and the pulley gear and the grip pulley rotate in the second direction opposite to the first direction, and
the wire wound in the second direction is wound around the grip pulley and the wire wound in the first direction is unwound from the grip pulley, in response to the rotation of the grip pulley, so as to cause a rotational motion of the first jaw or the second jaw.

19. The surgical instrument of claim 16,
wherein the first jaw wire comprises a first strand and a second strand as a pair of wires,
the second jaw wire comprises a third strand and a fourth strand as a pair of wires,
the first strand is wound around the grip pulley in a first direction, and the second strand is wound in a second direction opposite to the first direction, and
the third strand is wound around the grip pulley in the first direction, and the fourth strand is wound in the second direction opposite to the first direction.

20. The surgical instrument of claim 9,
wherein when the grip lever rotates, a rotating force transmitted to the grip pulley and a rotation angle displacement of the grip pulley vary depending on a ratio of a diameter of the pulley gear to a diameter of the grip gear.
